(19) United States
(12) Patent Application Publication
(10) Pub. No.: US 2016/0067711 A1
(43) Pub. Date: Mar. 10, 2016

(54) SYSTEMS AND METHODS FOR SINGLE CELL ISOLATION AND ANALYSIS

(71) Applicant: THE REGENTS OF THE UNIVERSITY OF MICHIGAN, Ann Arbor, MI (US)

(72) Inventors: Euisik Yoon, Superior Township, MI (US); Yu-Chih Chen, ANN ARBOR, MI (US)

(21) Appl. No.: 14/848,820

(22) Filed: Sep. 9, 2015

Related U.S. Application Data
(60) Provisional application No. 62/047,819, filed on Sep. 9, 2014.

(51) Int. Cl. B01L 3/00; C12M 1/00; C12M 3/06

(52) U.S. Cl. CPC B01L 3/502761; C12M 23/16; C12M 23/20; B01L 2200/0668; B01L 2300/0893; B01L 2300/0654; B01L 2400/049; B01L 2300/12; B01L 2300/0663

(57) ABSTRACT

The present disclosure relates to devices, systems, and methods for single cell isolation and analysis. In particular, the present disclosure relates to systems and methods for isolating single cells present at low numbers.

The present application claims priority to U.S. Provisional Patent Application Ser. No. 62/047,819, filed Sep. 9, 2014, the disclosure of which is herein incorporated by reference in its entirety.

### FIELD OF THE INVENTION

The present disclosure relates to devices, systems, and methods for single cell isolation and analysis. In particular, the present disclosure relates to systems and methods for isolating single cells present at low numbers.

### BACKGROUND OF THE INVENTION

Cancer cell heterogeneity is one of key challenges in modern cancer study. Due to the genomic instability of cancer cells (Negrini et al., (2010) Nat Rev Mol Cell Biol. 11(3):220-228), certain cells may have higher capability of drug resistance, metastasis and tumorgenesis (Visvader and Lindeman G J (2008) Nat Rev Cancer. 8(10):755-768). Studying these sub-populations separately can lead to effective therapeutic targets. For example, the state transition, such as the epithelial-to-mesenchymal transition (EMT) is one of key events in the tumor development and metastasis (Yang and Weinberg R A. (2008) Dev Cell. 14(6):818-29).

These mechanisms cannot be easily studied by conventional dish-based assays. Recent development of microfluidics has provided single-cell assay capability by isolating and culturing cells in an array of microchambers (Chung et al., Appl. Phys. Lett, 98(12), 3701 (2011)). However, these methods lack the ability to retrieve a target single cell for further analysis (e.g., genotyping) and assays (e.g., drug-screening).

Conventional cell detachment schemes, such as trypsinization or PNIPAAm-based detachment (Canavan et al, (2005) J Biomed Mater Res A. 75(1):1-13) do not provide any spatial resolution; they give blank detachment of entire cells from the substrate. The PALM CombiSystem developed by Zeiss can detach cells adhered on a laser absorbing film. However, detaching cells from the special film limits spatial resolution and it is difficult to handle cells over the film. On top of that, the cell detachment based on photodegradation of the substrate film, which generates acid, may lead to toxicity to the cells (Kimio et al., Proceedings of MicroTAS 2013 100-102).

Recently, an IR-triggered detachment method of single cells on CNT substrates was reported (Sada et al, (2011) ACS Nano. 5(6):4414-21). However, cell viability was poor because of heat-induced cell necrosis under direct laser irradiation. Recently, cell detachment using ultrasound-induced cavitation was demonstrated (Baac et al., (2012) Sci. Rep. 2, 989), but unfortunately this approach only works on Petri dishes and is not compatible with microfluidic arrangement due to acoustic attenuation by PDMS.

It is believed that fibroblasts are a major micro-environmental regulator in cancer and are critical in tumorigenesis and metastasis (Raghu Kalluri and Michael Zeisberg, Nature Reviews Cancer, 2006, 6, 392-401; Dan Liu, and Peter J. Hornsby Cancer Res 2007, 67(7), 3117-26). Recently, it was demonstrated that the co-transplant of cancer cells and fibroblast can boost the formation of cancer and that cancer associated fibroblast can skew the differentiation or de-differentiation of cancer stem cells (Raghu Kalluri and Michael Zeisberg, Nature Reviews Cancer, 2006, 6, 392-401; Dan Liu, and Peter J. Hornsby Cancer Res 2007, 67(7), 3117-26). As one expects different regulation of CSC sternness with fibroblast co-culture, a co-culture platform that can maintain clonal spheres while providing signaling from adherent stromal cells is needed.

### SUMMARY OF THE INVENTION

The present disclosure relates to devices, systems, and methods for single cell isolation and analysis. In particular, the present disclosure relates to systems and methods for isolating single cells present at low numbers.

Embodiments of the present disclosure provide a system, comprising: a) a cell capture device comprising a plurality of wells, wherein each well is configured to capture a single cell, and wherein each well comprises a transport channel, a cell culture chamber and a transport channel; b) a fluid transport device configured to transport fluid through the transport channel of the cell capture device without dead volume. In some embodiments, the cell capture device comprises at least 100 wells (e.g., at least 1000 wells, at least 5000 wells, or at least 10,000 wells). In some embodiments, the system further comprises a microscope in optical communication with the cell capture device. In some embodiments, the fluid transport device is a vacuum device. In some embodiments, the vacuum device is separated from the cell culture chamber at a distance of approximately 50 μm (e.g., 50 μm). In some embodiments, the cell capture device is constructed of PDMS. In some embodiments, the cell culture well is approximately 100-500 μm in length and width, the main channel is approximately 50 μm in width, and the device is approximately 40 to 100 μm in height. In some embodiments, the system is configured to capture 10 or less cells present in a volume of 10 μl or less. In some embodiments, the cell culture chamber further comprises one or more sensors for sensing pH, oxygen, glucose, proteins, or metabolic byproducts.

Further embodiments provide a method of capturing single cells, comprising: a) contacting a solution comprising one or more cells with a system comprising a cell capture device comprising a plurality of wells, wherein each well is configured to capture a single cell, and wherein each well comprises a transport channel, a cell culture chamber and a transport channel and a fluid transport device configured to transport fluid through the transport channel of the cell capture device without dead volume; and b) transporting the solution through the system such that the cells are captured in the wells. In some embodiments, the method further comprises the step of contacting the cells with a test compound (e.g., a drug). In some embodiments, the method further comprises the step of performing a cell secretion, cell metabolism, or proteolytic activity assay on the cells. For example, in some embodiments, the proteolytic activity of single cells is assayed.

Additional embodiments provide a cell culture device, comprising a plurality of chambers, wherein at least one of each of the chambers comprises an adherent culture chamber configured for cells to adhere to, a suspension culture chamber configured to prevent adherence of cells, and a plurality of interaction channels. In some embodiments, interaction channels are an approximately 1.4 μm thick gap channel that connects the adhesion chamber to the suspension chamber. In some embodiments, interaction channels comprise microposts. In some embodiments, the cell capture device is constructed of PDMS (e.g. optionally fused to glass slides). In some embodiments, the PDMS is cured at room temperature. In some embodiments, the suspension culture chamber is coated in polyyHEMA. For example, in some embodiments, the polyyHEMA is stamped and then reflowed at a temperature of between 100 and 200° C. In some embodiments, plasma etch is used to remove residual polyyHEMA from the substrate. In some embodiments, the polyHEMA layer is selectively patterned on the suspension culture chamber. In some embodiments, one or more of the adherent culture chamber, suspension culture chamber, and interaction channels comprises one or more sensors for sensing pH, oxygen, glucose, proteins, or metabolic byproducts. In some embodiments, the device comprises at least 10 chambers (e.g., at least 100 chambers or at least 150 chambers). In some embodiments, devices further comprise addressable valves (e.g., for selective retrieval of cells or cell clusters). In some embodiments, devices further comprise sealing valves to isolate the suspension chamber (e.g., for in situ analysis). In some embodiments, the adherent culture chamber comprises a transwell and said suspension culture chamber comprises a plurality of microwells (e.g., coated in polyHEMA or Pluronic F108 surfactant). In some embodiments, the suspension culture chamber and/or the adherent culture chamber comprise a plurality of electronic bar codes (e.g., one per microwell). In some embodiments, the electronic bar codes are bar code patterns for automatic (e.g., computer-aided) tracking/monitoring.

Additional embodiments provide a method, comprising: a) contacting a cell culture device, comprising a plurality of chambers, wherein each of the chambers comprises an adherent culture chamber configured for cells to adhere to, a suspension culture chamber configured to prevent adherence of cells, and a plurality of interaction channels with at least a first solution comprising a first cell type such that the adherent culture chamber and the suspension culture chamber comprise at least one cell; and b) culturing the cells. In some embodiments, a second cell type is utilized. The present disclosure is not limited to a particular cell type. In some embodiments, first cell type is a cancer stem cell and the second cell type is a fibroblast. In some embodiments, cells from the adherent culture chamber and the suspension culture chamber interact in the interaction chamber. In some embodiments, secreted factors from the first or second cell types flow through the interaction channels and contact the first or second cell type.

Additional embodiments will be apparent to persons skilled in the relevant art based on the teachings contained herein.

### DESCRIPTION OF THE DRAWINGS

FIG. 1 shows operation of an exemplary single cell capture device.

FIG. 2 shows an example of single cell capture by an exemplary device.

FIG. 3 shows operation of an exemplary single cell capture device.

FIG. 4 shows the distribution of cells per well when loading 100 cells in 250 chambers.

FIG. 5 shows the distribution of cells per well with different cell concentration.

FIG. 6 shows isolation and re-open of an exemplary cell culture chamber.

FIG. 7 shows a microfluidic chip for single cell migration: (A) 3D schematics of a co-culture chamber having inner suspension culture chamber, outer adherent chamber and interacting channel connecting them, (B) photograph of a fabricated device having 120 chambers within 8 mm by 10.5 mm core area, and (C) micrograph showing a fabricated co-culture chamber (scale bar: 100 μm).

FIG. 8 shows fabrication process of the adhesion/suspension culture chip.

FIG. 9 shows a fabricated non-adherent microwell: (A, B) SEM of microwell before and after filling polyHEMA and (C, D) surface profile of microwell before and after filling polyHEMA measured by LEXT. PolyHEMA was measured to be 4 μm thick in the center. (scale bar: 100 μm)

FIG. 10 shows a PolyHEMA over-etched indented chamber. The PDMS substrate exposes at the center of the chamber.

FIG. 11 shows that T47D cells grow on: (A) Petri dish (all adherent), (B) micro-well without coating (all adherent), (C) surface coated with polyHEMA (all suspension) and (D) selectively polyHEMA coated substrate (suspension in the microwell but adherent elsewhere). (scale bar: 100 μm)

FIG. 12 shows flow simulations of the cell capture design: (a) the pressure distribution, (b) the flow velocity of the xy-plane, and (c) the flow velocity of the xz-plane.

FIG. 13 shows cell loading: (A) C2C12 cells loaded in the outer culture chamber on day 0, (B) C2C12 cells grew to monolayer on day 1, (C) single T47D cell (green fluorescent labelled) loaded in the inner chamber on day 1.

FIG. 14 shows the number of captured cell per chamber: (a) 50 cells loaded into a device with 120 chambers and (N=4) (b) 100 cells loaded into a device with 120 chambers (N=4). The experiment results match well with the Poisson distribution.

FIG. 15 shows simulations of cell secretion concentration in the chamber: (a) the distribution of cancer cell secretion, while the media flows from inner chamber to outer ring and (b) the distribution of fibroblast cells secretion, while the media flows from outer ring to inner chamber.

FIG. 16 shows differential cancer (T47D) sphere formation with and without co-culture with C2C12 cells: Representative cancer sphere on day 14 (A) without C2C12 or (B) co-cultured. (C) Sphere formation rate with and without co-culture after 14-day culture. (N=4), ** P<10−2. (d) Average sphere size with and without co-culture (N=4), ** P<10−2.

FIG. 17 shows a two-step cell loading process of devices of embodiments of the present disclosure.

FIG. 18 an exemplary microfluidic chip for cell-cell interaction.

### DEFINITIONS

To facilitate an understanding of the present invention, a number of terms and phrases are defined below:

As used herein, the term “dead volume” refers to physical space (e.g., in a microfluidics device) that is not part of a functional component of the device. In general, fluid in the dead volume or “dead space” of a device is not captured or analyzed. In some embodiments, “dead volume” is part of a loading channel or other unused portion of a device.

As used herein, the term “adherent culture chamber” refers to a well or chamber configured for cells to adhere to. In some embodiments, adherent culture chambers are adherent due to the surface material or coating.

As used herein, the term “suspension culture chamber” refers to a chamber or well that cells are unable to adhere to. In some embodiments, the surface of the suspension culture chamber is coated with a material that prevents or repels cells (e.g., polyHEMA).

The term “sample” is used in its broadest sense. On the one hand it is meant to include a specimen or culture. On the other hand, it is meant to include both biological and environmental samples. A sample may include a specimen of synthetic origin.

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

### DETAILED DESCRIPTION OF THE INVENTION

The present disclosure relates to devices, systems, and methods for single cell isolation and analysis. In particular, the present disclosure relates to systems and methods for isolating single cells present at low numbers.

In a conventional cell loading scheme, either driving by pump or gravity flow, the dead volume, which never flows into the microfluidics, is at least several microliters. When the sample size is comparable to the dead volume, the dead volume will cause significant loss of the sample. Also, the hydrodynamic capture scheme is not 100% capture rate, it suffers from another loss. Due to these reasons, although microfluidics claim to work with small number of cell, operating sample smaller than 100 cells is not really practical. However, the number of isolated circulating tumor cell, which accounts for the distant metastasis is typically less than 100 or even less than 10 cells in a test sample.

The capability to use small sample is one of key challenges in modern cancer study and personalized medicine. Conventional (dish based) approaches need hundreds of thousands cells for an assay. A patient biopsy sample may be barely enough for a trial and it is impractical to screen multiple drugs and vary different dosage for personalized medicine. As getting a biopsy is a painful process, it can be difficult to persuade the patient to get even one biopsy sample. For this reason, circulating tumor cells (CTC), which can be obtained from a patient's blood is a promising alternative. However, CTCs are very rare, and typically only 10-100 cells can be obtained from 7.5 mL patient blood. Hence, there is an outstanding need for the capability to handle small samples.

Compared to conventional dish based approaches, microfluidics, which works with small volume of liquid, has an intrinsic advantage of manipulating small sample. Instead of using millions of cells, microfluidics does a good job in the range of thousands of cells. Although high single cell capture rate (number of captured cells/number of chambers) can be optimized up to 80-90%, the cell capture efficiency (number of captured cells/number of cells used) is typically low (<10%). To capture single cells by hydrodynamic, DEP or other techniques, a large amount of cells are lost in sample preparation and cell capturing process. It is impractical to work with 10-100 cells for an assay.

Accordingly, embodiments of the present disclosure provide devices, systems, and methods for isolating a low number of rare cells from a population. Embodiments of the present disclosure provide microfluidics devices and systems that efficiently capture ultra-small samples down to 10 cells in a several μL solution. In some embodiments, the cells are cultured in a chamber for down-stream analysis, study of cell-cell interaction, etc. In some embodiments, cells are removed from the chambers for further analysis.

The systems and methods of the present disclosure provide the advantages of high capture efficiency of small samples. Previously, it was almost impossible to perform assays on 10 or fewer cells in a sample. However, experiments describe herein demonstrated that exemplary devices were able to capture 7 single cells out of 10 cells, and 7 assay data points can be obtained from an ultra-small sample of 10 cells.

The devices described herein provide easy and reliable isolation of cell from chambers by flowing oil in the main channel. Compared to conventional pneumatic valving, which requires two layer fabrication and complex control, the devices of embodiments of the present disclosure can isolate cell capture chambers reliably by immiscible two-phase isolation. The devices further provide the capability to do perform cell-cell interaction studies from a small sample and to monitor the secretion profile and metabolism of single cells from a small sample.

Embodiments of the present disclosure provide a variety of different chamber sizes designed for different cell concentration or sizes (e.g., to allow beads, embryos, fertilized eggs, eggs, follicles, etc. to be captured).

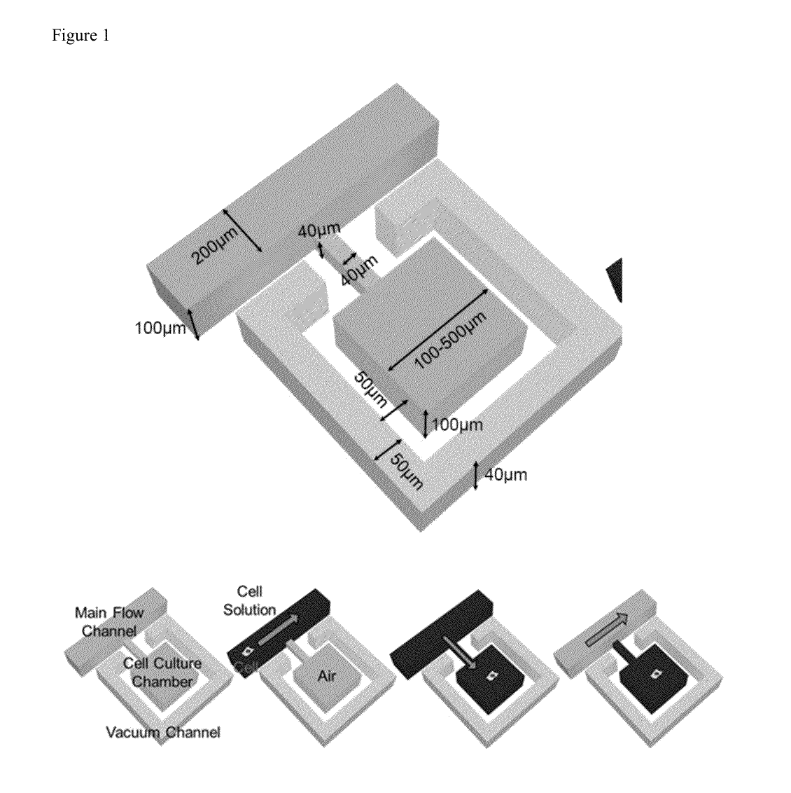

For example, in some embodiments, the present disclosure provides single cell capture systems and devices. In some embodiments, the devices comprise a plurality of wells (e.g., at least 100, at least 200, at least 300, at least 400, at least 500, at least 1000, at least 5000, at least 10,000, etc.). In some embodiments, each well (See e.g., FIG. 1) comprises a main flow channel, a cell culture chamber, and a transport (e.g., vacuum) channel. In some embodiments, the cell culture well is approximately 100-500 μm in length and width, the main channel is approximately 50 μm in width, and the device is approximately 40 to 100 μm in height, although other sizes are contemplated.

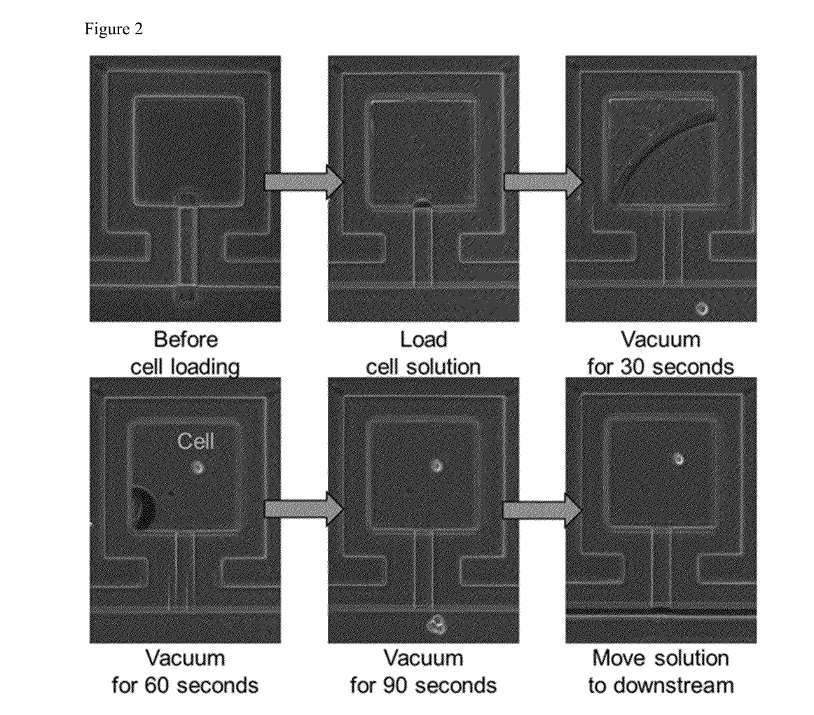

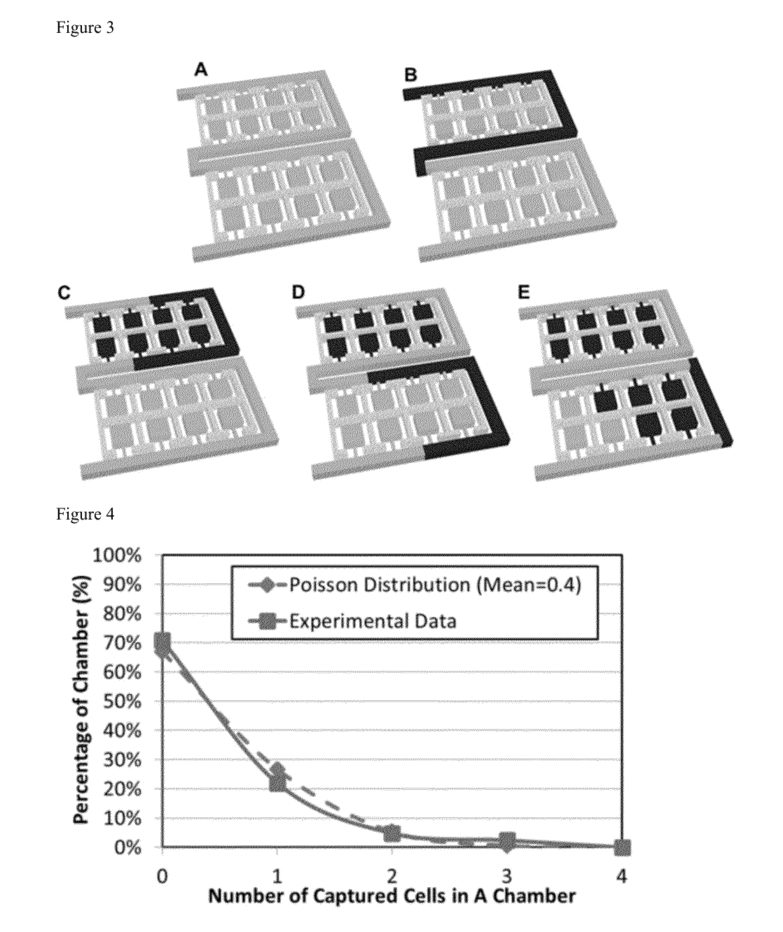

In some embodiments, the devices are configured to minimize or eliminate dead volume to improve capture efficiency of rare or low concentration cells. In some embodiments, a vacuum or other transport component is used to draw the entire sample volume into the device to maximize capture of rare cells. In some embodiments, the loading process (e.g., as shown in FIGS. 2-3) is repeated to capture additional cells. In some embodiments, the devices described herein are suitable for capture of as few as 10 cells (e.g., 1, 2, 3, 4, 5, 6, 7, 8, 9, 10, 15, 20, etc.) in a volume of several microliters (e.g., less than 10 μl, less than 9 μl, less than 8 μl, less than 7 μl, less than 6 μl, less than 5 μ, less than 4 μl, less than 3 μl, less than 2 μl, less than 1 μl, etc.). In some embodiments, systems comprise a microscope for identifying which wells comprise a single cell and confirming the identity of captured cells.

In some further embodiments, the present disclosure provides devices for analysis of cells in a co-culture environment. In some embodiments, the co-culture wells are integrated into the single cell capture devices described herein or other microfluidic devices.

Although cancer-stromal interactions are critical in tumorigenesis, conventional dish based co-cultures lack the capability to study cell heterogeneity by tracking single cell behavior (Raghu Kalluri and Michael Zeisberg, Nature Reviews Cancer, 2006, 6, 392-401; Dan Liu, and Peter J. Hornsby Cancer Res 2007, 67(7), 3117-26). To understand a heterogeneous population such as cancer, ideal co-culture platforms should be able to provide single cell resolution for characterizing individual cell behavior rather than the averaged behavior (Gupta, C. L. Chaffer and R. A. Weinberg, Nat. Med., 15(9), 1010-1012 (2009)). There are a number of previous works reporting on microfluidic platforms for cell-to-cell interaction studies (M. Heneweer, M. Muusse, M. Dingemans, P. C. de Jong, M. van den Berg, J. T. Sanderson, Toxicol Sci., 2005, 83(2), 257-63; A. Y. Hsiao, Y. S. Torisawa, Y. C. Tung, S. Sud, R. S. Taichman, K. J. Pienta, S. Takayama, Biomaterials, 2009, 30(16), 3020-7; J. Park, H. Koito, J. Li, A. Han, Biomed Microdevices, 2009, 11(6), 1145-53; M. Bauer, G. Su, D. J. Beebe, A. Friedl, Integr Biol., 2010, 2(7-8):371-8; H. Ma, T. Liu, J. Qin, B. Lin, Electrophoresis, 2010, 31(10), 1599-605; Y. Gao, D. Majumdar, B. Jovanovic, C. Shaifer, P. C. Lin, A. Zijlstra, D. J. Webb, D. Li, Biomed Microdevices, 2011; 13(3):539-48; D. Majumdar, Y. Gao, D. Li, D. J. Webb, J. Neurosci Methods, 2011, 196(1), 38-44; E. Tumarkin, L. Tzadu, and E. Kumacheva, Integr. Biol., 2011, 3, 653-662; P. Ingram, Y. J. Kim, T. Bersano-Begey, X. Lou, A. Asakura, and E. Yoon, Proceedings of MicroTAS, Groningen, 2010, 277-279; J. Frimat, M. Becker, Y. Chiang, and J. West, Lab Chip, 2011, 11, 231-237; S. Hong, Q. Pan and L. P. Lee, Integr. Biol., 2012, 4, 374-80; Y.-C. Chen, Y.-H. Cheng, H. S. Kim, P. N. Ingram, J. E. Nor and E. Yoon, Lab Chip, 2014, 14 (16), 2941-2947).

Most devices require loading hundreds or thousands of cells in a device; thus, they still lack single cell resolution. Droplet based technology can provide a high-throughput combinatorial pairing of cells, but it lacks capabilities for long-term cell culture, limiting its applications in practical co-culture assays. Recently, several microfluidic devices reported cell pairing and cell-to-cell interaction at single-cell resolution, but those works were limited to adherent cell co-culture. For applications in cancer biology, 3D suspension culture is believed to maintain the stemness of CSCs, but suspension environments are too harsh for most adherent stromal cells (e.g. fibroblast cells, endothelial cells) (Gabriela Dontu, Wissam M. Abdallah, Jessica M. Foley, et al. Genes Dev. 2003 17: 1253-1270; Yi-Chung Tung, Amy Y. Hsiao, Steven G. Allen, Yu-suke Torisawa, Mitchell Ho and Shuichi Takayama, Analyst, 2011, 136, 473-478). Experiments described herein resulted in the development of a co-culture platform combining both suspension and adhesion culture in close proximity inside the same chamber. Compared to previous single-cell platforms (W.-H. Tan and S. Takeuchi Proc Natl Acad Sci U S A, 2007, 104(4), 1146-1151; Alison M Skelley, Oktay Kirak, Heikyung Suh, Rudolf Jaenisch and Joel Voldman., Nature Methods, 2009, 6, 147-152; J. Chung, Y.-J. Kim and E. Yoon, Appl. Phys. Lett, 98(12), 3701 (2011); Y.-C. Chen, P. Ingram, X. Lou, and E. Yoon, Proceeding of MicroTAS, 2012, 1241-1244), the presented platform also specializes in high capture efficiency, up to 75% of the loaded cells, even for small samples (50 cells). This enables the study of rare cell populations such as samples taken from primary cells.

The dual chamber design described herein is suitable for co-culture of multiple (e.g., two or more) types of cells with the following advantages: (1) capability to provide different culture environments for different cell types in the same chamber, (2) interaction of cells through narrow channels but avoiding cell migration into one side to the other, (3) control of flow direction for interaction during culture, and (4) handling of small samples with high cell capture efficiency.

Compared to the conventional polyHEMA coating technique, the techniques described herein provide a thinner (e.g., by spin-coating) and more uniform (e.g., by reflow) layer of polyHEMA. In addition, the polyHEMA patterning techniques, including selectively filling the indented well with polyHEMA and stamping polyHEMA, generate non-adherent culture environments with precise resolution (˜10 μm).

In some embodiments, wells are filled with a material with a different Young's modulus, or different material properties. In addition to providing adhesion/suspension environments, this technique provides soft/rigid cell-culture microenvironments. For example, in some embodiments, co-culture devices comprise an adherent culture chamber configured for cells to adhere to, a suspension culture chamber configured to prevent adherence of cells, and a plurality of interaction channels as shown in FIG. 1. In some embodiments, cells interact directly in the interaction channels, while in some embodiments, secreted factors from one cell type interact with another cell type. In some embodiments, interaction channels are an approximately 1.4 μm thick gap channel that connects the adhesion chamber to the suspension chamber. In some embodiments, interaction channels comprise microposts. In some embodiments, the microposts prevent PDMS collapse and but also maintain high effective flow cross-sectional area, hence small flow resistance useful for the cell loading process.

In some embodiments, devices further comprise addressable valves (e.g., for selective retrieval of cells or cell clusters). In some embodiments, devices further comprise sealing valves to isolate the suspension chamber (e.g., for in situ analysis).

The present disclosure is not limited to particular methods for fabricating microfluidic devices. In some embodiments, devices are made by the sandwiching of three layers (e.g., poly-dimethylsiloxane (PDMS) layers). In some embodiments, the top and bottom layers contain the main network of microfluidic channels. In some embodiments, the middle layer is a thin membrane.

In some embodiments, layers are made by supplying a negative “master” and casting a castable material over the master. Castable materials include, but are not limited to, polymers, including epoxy resins, curable polyurethane elastomers, polymer solutions (e.g., solutions of acrylate polymers in methylene chloride or other solvents), curable polyorganosiloxanes, and polyorganosiloxanes which predominately bear methyl groups (e.g., polydimethylsiloxanes (“PDMS”)). Curable PDMS polymers are well known and available from many sources. Both addition curable and condensation-curable systems are available, as also are peroxide-cured systems. All these PDMS polymers have a small proportion of reactive groups which react to form crosslinks and/or cause chain extension during cure. Both one part (RTV-1) and two part (RTV-2) systems are available. Additional curable systems are preferred when biological particle viability is needed.

In some embodiments, transparent devices are desirable. Such devices may be made of glass or transparent polymers. PDMS polymers are well suited for transparent devices. A benefit of employing a polymer which is slightly elastomeric is the case of removal from the mold and the potential for providing undercut channels, which is generally not possible with hard, rigid materials. Methods of fabrication of microfluidic devices by casting of silicone polymers are well known. See, e.g. D. C. Duffy et al., “Rapid Prototyping of Microfluidic Systems in Poly(dimethylsiloxane),” Analytical Chemistry 70, 4974-4984 (1998). See also, J. R. Anderson et al., Analytical Chemistry 72, 3158-64 (2000); and M. A. Unger et al., Science 288, 113-16 (2000), each of which is herein incorporated by reference in its entirety.

In some embodiments, glass slides are bonded to the back (e.g., outside) of the PDMS substrate (e.g., to minimize surface distortion of the PDMS). In some embodiments, room temperature cured PDMS is utilized to eliminate any potential thermal mismatch between multiple layers of PDMS used in fabrication.

In some embodiments, fluids are supplied to the device by any suitable method. Fluids may, for example, be supplied from syringes, from microtubing attached to or bonded to the inlet channels, etc.

Fluid flow may be established by any suitable method. For example, external micropumps suitable for pumping small quantities of liquids are available. Micropumps may also be provided in the device itself, driven by thermal gradients, magnetic and/or electric fields, applied pressure, etc. All these devices are known to the skilled artisan. Integration of passively-driven pumping systems and microfluidic channels has been proposed by B. H. Weigl et al., Proceedings of MicroTAS 2000, Enshede, Netherlands, pp. 299-302 (2000).

In other embodiments, fluid flow is established by a gravity flow pump, by capillary action, or by combinations of these methods. A simple gravity flow pump consists of a fluid reservoir either external or internal to the device, which contains fluid at a higher level (with respect to gravity) than the respective device outlet. Such gravity pumps have the deficiency that the hydrostatic head, and hence the flow rate, varies as the height of liquid in the reservoir drops. For many devices, a relatively constant and non-pulsing flow is desired.

To obtain constant flow, a gravity-driven pump as disclosed in published PCT application No. WO 03/008102 A1 (Jan. 18, 2002), herein incorporated by reference, may be used. In such devices, a horizontal reservoir is used in which the fluid moves horizontally, being prevented from collapsing vertically in the reservoir by surface tension and capillary forces between the liquid and reservoir walls. Since the height of liquid remains constant, there is no variation in the hydrostatic head.

Flow may also be induced by capillary action. In such a case, fluid in the respective outlet channel or reservoir will exhibit greater capillary forces with respect to its channel or reservoir walls as compared to the capillary forces in the associated device. This difference in capillary force may be brought about by several methods. For example, the walls of the outlet and inlet channels or reservoirs may have differing hydrophobicity or hydrophilicity. Alternatively, the cross-sectional area of the outlet channel or reservoir is made smaller, thus exhibiting greater capillary force.

In some embodiments, flow is facilitated by embedded capacitor valves that pump fluids in a separate channel when pressurized. This is achieved by having a series of valves in the bottom that direct a pressurized gas or liquid causing the membrane to deform and squeeze the fluid in the top channel forward. Additional control is provided by having valves in the top layer that can open sequentially.

### II. Methods

Embodiments of the present disclosure provide methods of capturing, culturing, and analyzing single cells or colonies of cells. The present disclosure is not limited to a particular cell type. The systems and methods described herein find use with a variety of cell types, including prokaryotic and eukaryotic cells and single cell organisms. In some embodiment, human or mammal cells are utilized (e.g., primary cells, immortalized cells, cancer cell lines, cancer stem cells, etc.).

In some embodiments, live cells are analyzed. In some embodiments, intact fixed cells are analyzed. In some embodiments, cells are lysed and molecular analysis is performed.

In some embodiments, the method further comprises the step of performing a cell secretion, cell metabolism, or proteolytic activity assay on the cells. For example, in some embodiments, the proteolytic activity of single cells is assayed.

In some embodiments, the devices of embodiments of the present disclosure comprise micro-sensors formed by mixing sensors with polyHEMA or other polymers. In some embodiments, sensors are integrated into cell capture and/or culture wells of single cell capture or co-culture devices. In some embodiments, the polymer is stamp-patterned and placed inside a microfluidic chamber to monitor single cell metabolism.

The present disclosure is not limited to particular sensing dyes or other sensors. In some embodiments, one or more sensors for sensing pH, oxygen, glucose, proteins, or metabolic byproducts are utilized.

Two exemplary sensing options for in-situ sensors are:

electrical/impedance sensing and fluorescent sensing. In electrical sensing, a small (10-50 um square) piece of electrode is fabricated inside the chamber. In some embodiments, as the chamber size is approximately 200 um square, multiple electrodes performing different surface chemical reactions for sensing different metabolites can be multiplexed in the same chamber.

For fluorescent based sensing, a fluorescent dye that changes color/intensity under different pH, oxygen, glucose and metabolites is either printed by inkjet preinter, stamped, flow patterned, or patterned by lithography as a 10-50 um square inside the chamber. In some embodiments, dyes can be mixed with PolyHEMA or PEG for stamping. The sensor is much smaller than the chamber, enabling multiplex sensing. After cell loading, the small chamber is isolated either by oil or air, so no media/metabolites exchange occurs. Thus, secreted proteins or other chemicals are monitored by the sensor in-situ. As multiple (multiplexed) sensors for pH, oxygen, glucose, proteins, and metabolic byproducts can be placed in the same chamber, the metabolism of a single cell can be monitored.

Exemplary fluorescent glucose sensors, include, but are not limited to, Concanavalin A (Con A); Glucose oxidase and glucose dehydrogenase; Hexokinase/glucokinase; Bacterial glucose-binding protein; and Boronic acid derivatives and are reviewed in, for example, Pickup et al., Biosensors and Bioelectronics 20 (2005) 2555-2565; herein incorporated by reference in its entirety. Exemplary fluorescent pH sensors include, but are not limited to, BCECF (2′,7′-Bis(3-carboxypropyl)-5(6)-carboxyfluorescein): useful for pH 6.5-7.5 and HPTS (8-Hydroxypyrene-1,3,6-trisulfonic acid trisodium salt). Exemplary oxygen and carbon dioxide sensors are reviewed in, for example, Chu et al., Photonic Sensors (2011) Vol. 1, No. 3: 234-250; herein incorporated by reference in its entirety.

Examples of analytes further include, but are not limited to, acarboxyprothrombin;

acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-.beta. hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free .beta.-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, .beta.); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin; insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; nicotine; ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), and 5-hydroxyindoleacetic acid (FHIAA).

Exemplary sensors are described in, for example, U.S. Pat. No. 6,951,947 (2005); U.S. Pat. No. 7,176,037 (2007); U.S. Pat. No. 7,351,797 (2008): U.S. Pat. Nos. 7,592,188 (2009), 8,815,523, and Tolosa, L., et al., Glucose sensor for low-cost lifetime-based sensing using a genetically engineered protein. Anal Biochem, 1999. 267(1): p. 114-20; each of which is herein incorporated by reference in its entirety.

In some embodiments, molecular analysis is performed on cells. The present disclosure is not limited to particular types of analyses. Examples include, but are not limited to, screening cells for gene expression at the mRNA or protein level (e.g., via reporter genes in live cells or molecular analysis); screening compounds (e.g., drugs) for their effect on cell growth, cell death, viral infectivity, or gene expression; screening viruses for infectivity (e.g., plaque formation); and screening for mutations, copy number variation, methylation status, or polymorphisms (e.g., SNPs).

The present disclosure is not limited to particular analysis methods. Examples include, but are not limited to, sequencing analysis, methylation analysis, copy number variation analysis, protein analysis, hybridization analysis, and amplification analysis. Exemplary analysis methods are described herein.

A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts (e.g., those described in table 1) by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

Nucleic acids may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

In some embodiments, gene expression is detected by detected altered levels of polypeptides encoded by the genes (e.g., using immunoassays or mass spectrometry).

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays. Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

### EXAMPLES

The following examples are provided in order to demonstrate and further illustrate certain preferred embodiments and aspects of the present invention and are not to be construed as limiting the scope thereof.

### Example 1

Operation of the Small Sample Capture Device

The device eliminates dead volume by driving all of the cell solution into the microfluidics. FIG. 1 shows the operation of a single capture well. The design composes of a main-channel, which transports the cell solution, a cell culture chamber and vacuum channel, which drives the solution into the chamber. One useful characteristic of PDMS is its gas permeability, so the air can diffuse through the PDMS sidewall to the vacuum channel. As a result, the cell can be loaded into the chamber with the cell solution. FIG. 2 demonstrates a cell loading process. One cell was loaded into the chamber.

FIG. 3 illustrates the operation of the device. First, liquids are pulled downstream to drive the cell solution into the main channel. Then, the vacuum channel is sued to drive the solution and cells into the chamber. If there is any residual solution in the main channel as shown in FIG. 3(C), it is driven downstream and the process is repeated to load the remaining solution into the chamber. The process is repeated until all solution is driven into the chambers. With a sample of 10 cells in 1 μL, 7 wells with a single cell were obtained. In some embodiments, an automated cell capture scheme that utilizes a programmed pump and vacuum system is used.

Control of the Number of the Captured Cells

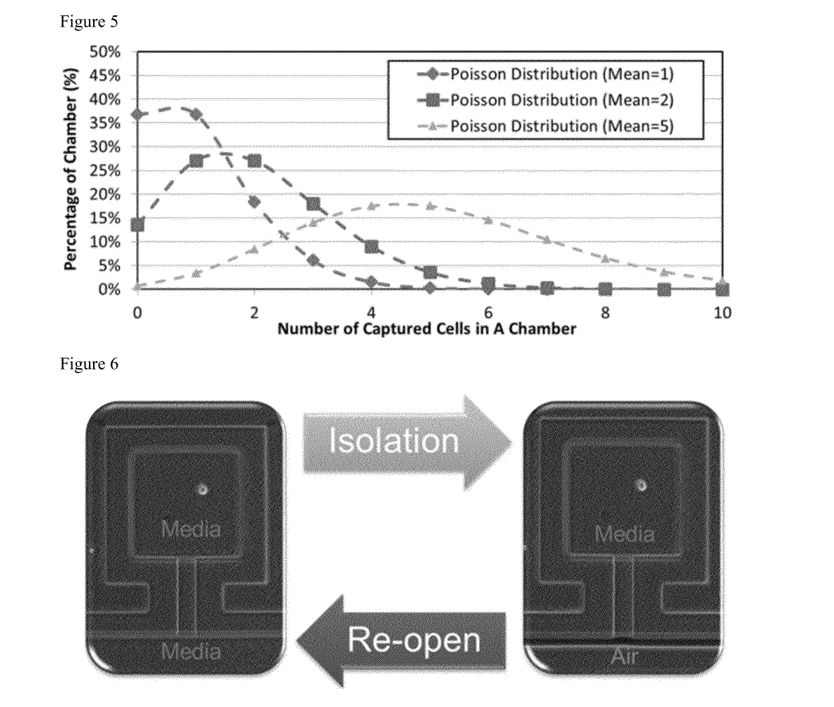

The number of cells per chamber was optimized to one. FIG. 4 demonstrates the loading result of loading 100 cells in 250 chambers. The experiment data matches well with the Poisson distribution. The number of cells per chamber is controlled by the cell concentration in the solution. When the cell concentration is higher, more cells per chamber are found. FIG. 5 shows the Poisson distribution in different concentrations. Using an appropriate concentration, one can load the desired number of cells into a chamber. Although not all chambers have exactly the desired number of cells, the majority of the distribution will have the desired number.

Cell-Cell Interaction in the Platform

Conventional cell-cell interaction works with many cells. However, these platforms cannot study the heterogeneity of single cell. The devices described herein, which provide single cell resolution, are able to study such heterogeneity. Cell pairs are isolated in a chamber for two reasons: to accumulate enough secreted proteins for interaction and to avoid the cross talk between chambers. Previously, isolation was implemented by pneumatic valving, but it requires a large area, and the two layer fabrication process is complex. In this work, the cell loading scheme and isolation scheme are combined. When the media is driven downward, the main channel is replaced by air. The cells in each chamber are isolated as shown in the FIG. 6. For reliable cell isolation, oil is used as an alternative to generate a two-phase isolation of captured cells. With this technique, cell-cell interaction is without cross-talk between chambers. After several hours, the culture media is re-injected into the main channel to refresh the culture media in the chamber and supply nutrition to the cells.

Single Cell Secretion and Metabolism

The metabolism and secretion of single cells is studied in situ. In-situ sensors are stamped, flow patterned, or patterned by lithography in the cell culture chamber. After isolation, the secretion of that single cell is diffused in the chamber, so the secreted protein or other chemicals is monitored by the sensor. Multiple (multiplexed) sensors for pH, oxygen, glucose, proteins, and metabolic byproducts can be placed in the same chamber, so the metabolism of that single cell is monitored.

### Example 2

Design and Fabrication

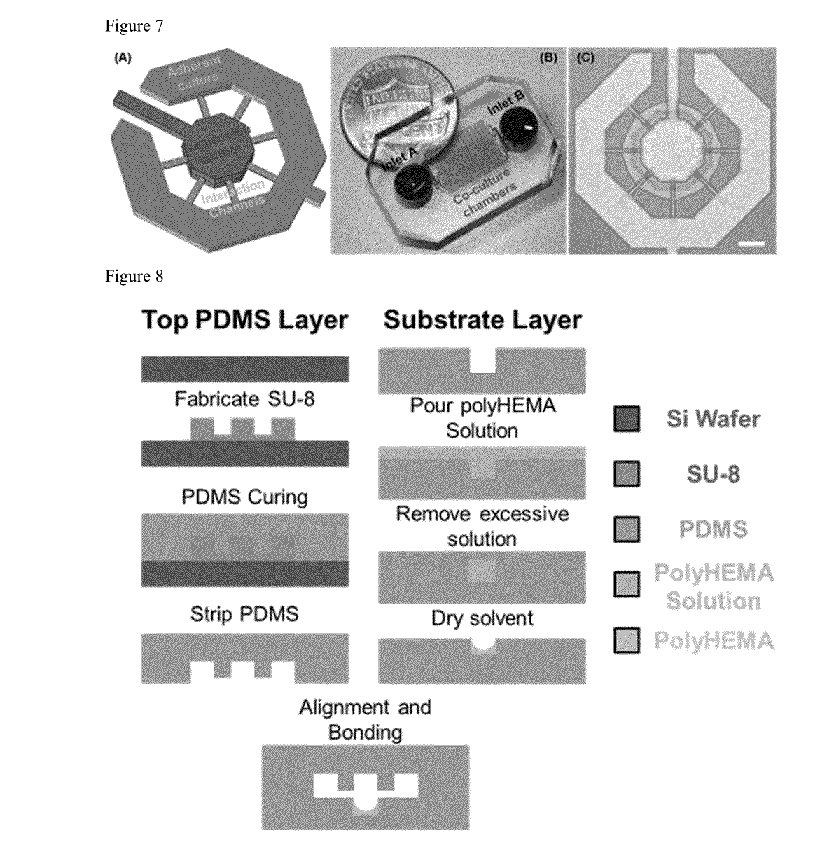

The co-culture platform is composed of an inner suspension culture chamber, an outer adherent culture chamber, and narrow interaction channels connecting them (FIG. 7(a)). The whole device comprises or consists of 120 co-culture units (15 by 8) (FIG. 7(b)), and each unit is composed of the two culture chambers connected by 7 narrow (cross-section 3 μm by 20 μm and 100 μm long) channels. To facilitate suspension and adherent culture on the same device, two layers of PDMS are used in fabrication. The bottom layer was patterned with indented microwells that were selectively coated with Poly-hydroxyethylmethacrylate (polyHEMA, Sigma-Aldrich), which has been extensively used as an adhesion blocking coating material [34]. The top channel layer is patterned with microfluidic channels for flow control and chambers for co-culture.

These two PDMS layers (channel layer and substrate layer) were separately fabricated using a soft lithography processes, and then aligned and bonded together, as shown in FIG. 8. For the channel layer, two masks were used to fabricate a SU8 (Microchem) master mold: the first mask for narrow interaction channels (3 μm height) and the second mask for main microfluidic channels and adherent cell culture chambers (40 μm height). One mask was used to fabricate the SU8 master mold for the substrate layer which has indented chambers (40 um depth) for suspension cell culture. To make the chambers non-adherent, polyHEMA was filled in the suspension culture chambers by a stamping process developed in our lab. The polyHEMA is in ethanol solution (60 mg/mL in 95% ethanol) and was coated on the substrate PDMS. A piece of blank PDMS was pressed on top to squeeze out the excess solution leaving the polyHEMA only in the indented micro-wells. To improve the coating quality, the indented PDMS substrate was plasma treated to increase the hydrophilicity. This causes the polyHEMA ethanol solution to deposit only to the patterned PDMS substrate while stamping. Then, the substrate and the blank PDMS stamp were put on a hot plate at 110° C. for 2 hours under pressure, in order to facilitate evaporation of ethanol through the PDMS layer. During the evaporation of ethanol, the polyHEMA layer was deposited in the suspension cell culture chambers. To remove the undesired residual polyHEMA on the surface, 30 seconds of 800 Watt plasma polymer etching was performed using the YES polymer striper (the expected etching depth is 0.3 μm). This resulted in a clean PDMS surface with polyHEMA in only the suspension culture chamber. The fabricated substrate was then aligned and bonded to the other PDMS fluidic layer that contains the outer chambers and interaction channels. The fabricated device is shown in FIG. 7(c).

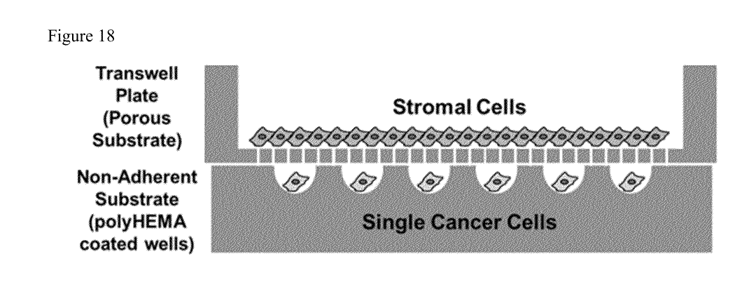

FIG. 18 shows an alternative of the cell-migration device of FIG. 7. In this device, non-adherent polyHEMA coated micro-wells are fabricated using the same technique described in FIG. 8. In some embodiments, Pluronic F108 Surfactant is used as an alternative to make the substrate non-adherent. Adherent culture is achieved in the transwell, and the suspension culture is performed in the polyHEMA coated well, as shown in FIG. 18. The transwell plate is placed on top of the micro-wells, so the cells can interact through the porous substrate of transwell plate. The microwell is not limited to particular sizes. In some embodiments, it is approximately 300 um deep and 200 um in diameter. In some embodiments, the well is deep enough so the cells will not be washed away when exchanging media. The substrate comprises at least 100 (e.g., at least 500, at least 1000, at least 5000, or at least 10,000) wells. In some embodiments, to hold the transwell plate at the right position, a circular pattern having the same geometry as the transwell plate is made on the substrate, so the transwell can insert into the substrate without movement. In some embodiments, barcoding (e.g., patterns that are automatically recognized) are used on each micro-well, so the cells can be automatically tracked using software.

Characterization of the Fabricated Surface

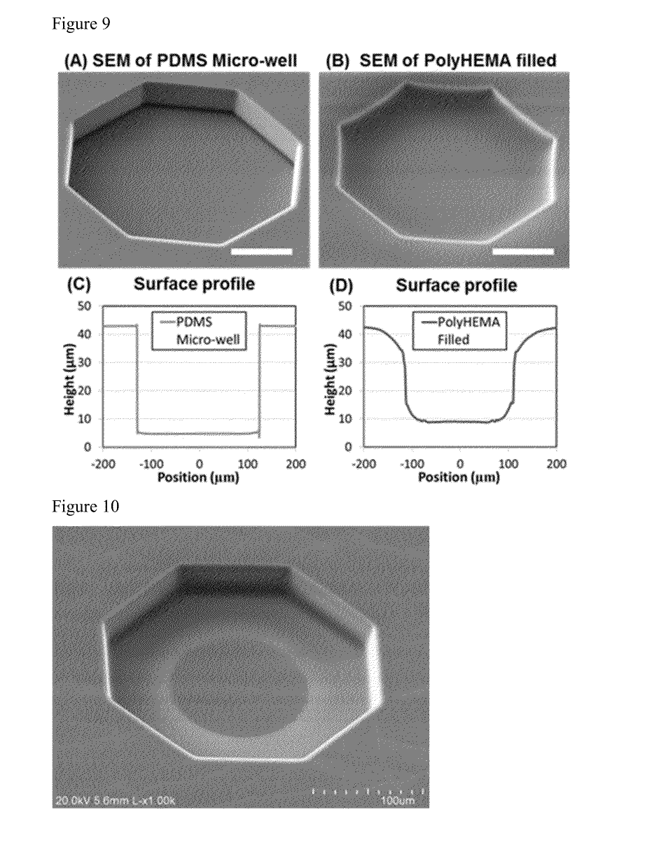

First, the surface profile of the fabricated substrates was analyzed by SEM. FIG. 9(a, b) shows the surface profile before the filling of polyHEMA. The vertical side wall of the indented micro-well is shown. After polyHEMA filling, the side wall of the indented micro-well becomes smooth. This indicates that polyHEMA was deposited on the substrate. If the polyHEMA is removed by plasma etching, the exposed PDMS on the bottom of the micro-well is shown in a SEM picture (FIG. 10). This indicator was used to determine the proper etching time. In addition, the surface profiles were measured using a laser interference microscope (LEXT, Olympus), as shown in FIG. 9(c, d)). The deposited polyHEMA covers the cross-section of the micro-well. Based on the comparison of profiles, the polyHEMA coating depth at the center of the chamber is approximately 4 μm, which is sufficient to generate a non-adherent culture surface.

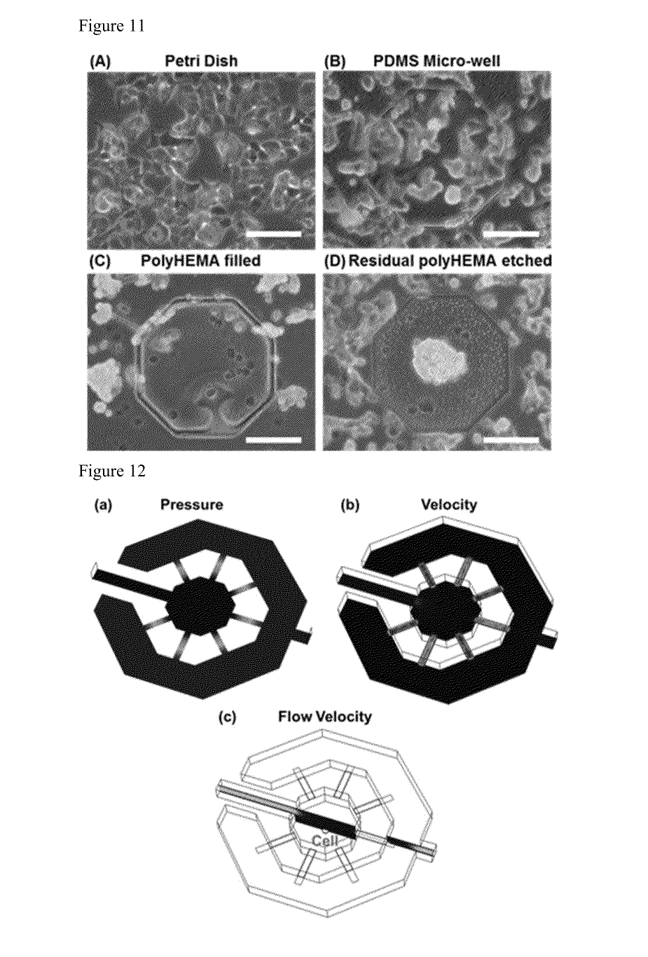

To verify the effect of polyHEMA coating, T47D breast cancer cells were cultured on the selectively coated substrate, compared with an uncoated PDMS substrate and a standard tissue culture plastic dish. (FIG. 11). Due to the difference of stiffness between PDMS (500 kPa) and the polystyrene (PS) dish (1 GPa), the observed cell morphologies were slightly different, but still, the cells were clearly attached on the PS dish and the uncoated PDMS substrate. The uncoated PDMS was patterned in the same way as the polyHEMA coated substrate (40 um deep wells), and the cells are adherent both inside and outside of the wells (FIG. 11 (b)). Without the plasma etching process, a thin polyHEMA layer is formed on the top surface of the polyHEMA coated substrate. As such in FIG. 11(c), cells remain rounded and aggregated on both the top surface and within the microwells, demonstrating that the T47D did not adhere. To attain dual suspension and adhesion culture on the same substrate, the residual polyHEMA was removed by the oxygen plasma etch. As the residual poylHEMA is much thinner (<0.5 μm) than the polyHEMA deposited in the wells (˜4 μm), the polyHEMA coating inside micro-wells can be preserved, while removing all polyHEMA on the non-indented surface. FIG. 11(d) demonstrates the desired selective coating behavior. As the well is non-adherent, cells formed aggregation in the well, while an adherent monolayer has grown on its surrounding area. In addition to T47D cell lines, multiple cell lines including C2C12 (mouse myoblast), MDA-MB-231 (breast cancer), and HCC38 (breast cancer) cells were cultured on the substrate. The selectivity was observed in all these cell lines, indicating that the fabrication process is robust and reliable for suspension/adherent cell culture.

Single Cell Isolation by Poisson's Distribution

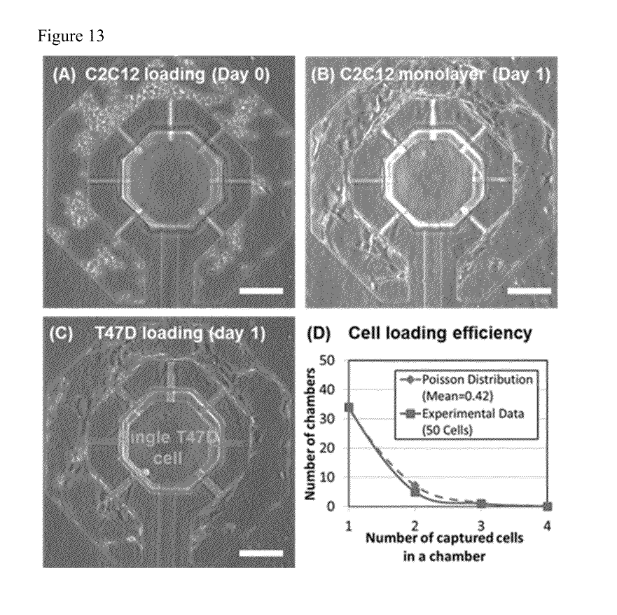

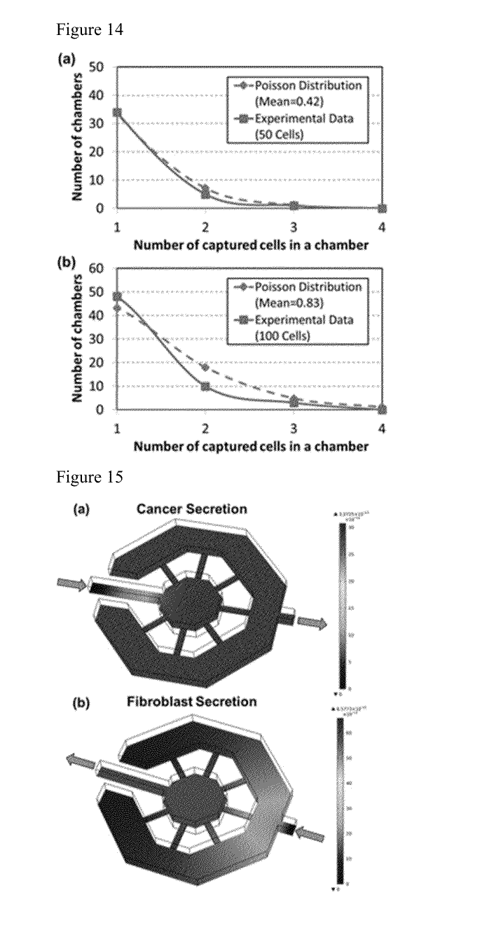

To show that cells cannot migrate between the inner and outer chambers, the interaction channels were designed to be 3 μm in height, preventing migration while allowing paracrine based interactions. During operation, the stromal cells in the outer ring are loaded first, and then allowed time to adhere to the substrate. The single cancer cells are then loaded and captured in the micro-well for suspension culture and single cell derived sphere formation. Once the single cell is loaded into the indented well, it will settle to the bottom of the well as demonstrated by the fluidic simulation shown in FIG. 12. Co-culture of T47D (breast cancer) and C2C12 (mouse fibroblast) cells to model tumor proliferation enhancement via fibroblast cell signaling was performed. The C2C12 cells, which utilize an adherent environment, were first loaded in the outer chamber (FIG. 13(a)). The cells were cultured with serum media (DMEM with 10% FBS), which contains attachment factors that encourage cell adhesion. After one day, cells were attached and grew to monolayer (FIG. 13(b)). As the interaction is much narrower than the size of cell, all coming single cancer cells were captured in the inner chambers (FIG. 13(c)). Although conventionally hydrodynamic capture schemes can have higher capture rates (60-90%), they are not ideal for small samples such as primary cells or CTCs, due to lower cell capture efficiency (typically less than 10%). Additionally, it is difficult to implement a hydrodynamic capture scheme in the platform due to the high fluidic resistance of the narrow interaction channels. Compared to the hydrodynamic capture, that may lose cells through the serpentine (by-pass) paths, a high capture efficiency was obtained in the presented platform by collecting all incoming cells into chambers. When the number of coming cells is much smaller than the number of the chamber, a single cell per chamber is obtained. Fifty cells were loaded in a device having 120 chambers, and 37 single cells were isolated in the chambers. The experimental results matched well with the Poisson distribution model (FIG. 14), showing that the capture scheme is suitable for the studies of small number (<100) of cells.

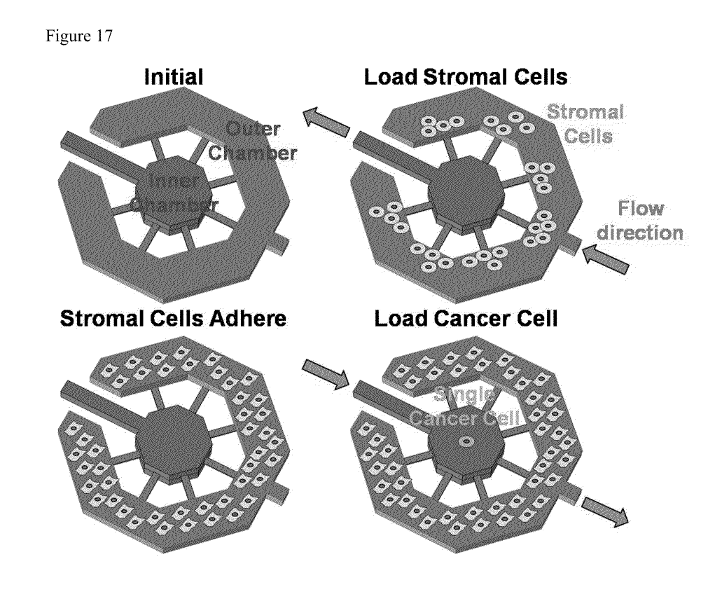

FIG. 17 shows a two-step cell loading process of the dual adherent-suspension micro-environment First, the adherent (e.g., stromal cells) are loaded in the outer ring. As the cross-section (2 um by 5 um) of interaction channel is much smaller than the size of typical mammalian cells, the stromal cells cannot penetrate into the inner chamber. As the interaction channels are uniformly distributed in the outer chamber, the loaded cells are uniformly distributed as well. After the loading of stromal cells, in some embodiments, a short period of time (e.g., one day) is allowed for cells to adhere. Then, the flow direction is reversed to load single suspension (e.g., cancer) cells. Once the single cell is loaded into the indented well, it will settle to the bottom of the well as demonstrated by the fluidic simulation.

Single cell Derived Sphere Formation Under the Influence of Stromal Cells

As shown in the simulations (FIG. 15), when media flows inward (from the outer ring to the inner suspension culture chamber), the media containing secretions from the fibroblasts can affect cancer cells located inside the inner chamber. Conversely, when media flows outward (from the inner suspension chamber to the outer ring culture chamber), the secretions from the cancer cell can affect the fibroblasts. When the flow is stopped, secreted factors from each population can diffuse throughout the chamber, allowing reciprocal interactions. As such, the platform can control the direction and type of interaction. An experiment was conducted in which the flow direction was alternated, flowing inward for 12 hours and outward for 12 hours during each 24-hour period.

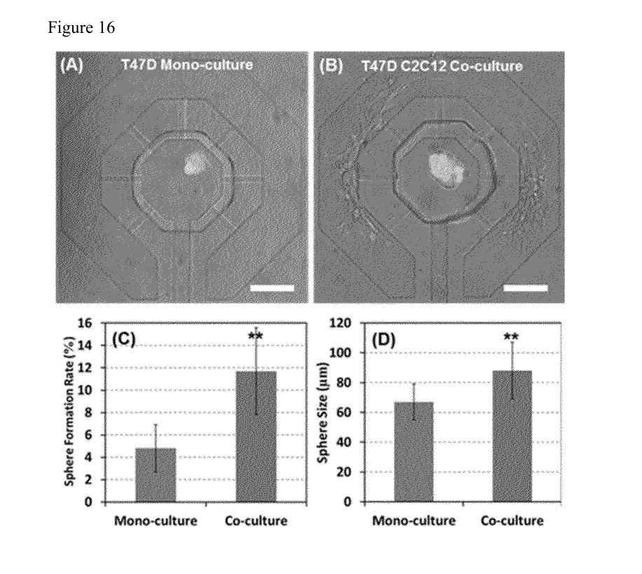

After co-culturing the two populations for 14 days, the interaction efficacy was quantitated by counting the number of single-cell-derived spheres present throughout the 120-well array [35, 36]. Compared to single cancer cells cultured without stromal interaction (FIG. 16 (a)), the cancer cells co-cultured with fibroblasts (FIG. 16 (b)) have doubled the sphere formation rate, indicating that fibroblast cells can boost the stem/progenitor cell potential in the cancer population (FIG. 16 (c)). Co-cultured spheres were observed to be larger (FIG. 16 (d)), indicating a higher proliferation rate as well. In summary, this experiment demonstrated high capture efficiency in a close-proximity dual suspension and adherent co-culture environment.

Other polyHEMA Coating Techniques

PolyHEMA Spin-Coating and Reflow

To achieve suspension culture, poly(2-hydroxyethyl methacrylate) (polyHEMA) was used as a non-adherent coating material. After absorbing water, polyHEMA forms many hanging long-chains that block cell adhesion on the substrate. Coating techniques are: polyHEMA is first dissolved in ethanol, and then the solution is added to a cell-culture dish. After natural evaporation of ethanol, polyHEMA forms a thin film on the dish. However, due to its surface roughness and non-uniformity, this process has two major challenges when integrated with microfluidics. First, large surface roughness, resulting from uncontrolled evaporation of ethanol, leads to poor bonding with the PDMS layer and induces severe leakage. Second, due to the non-uniformity of film thickness, a thicker polyHEMA coating (˜30 μm) is used to prevent pinholes or openings in the coating. As polyHEMA can absorb 50% water (w/w) and expand, the expanded polyHEMA hydrogel can block the fluidic channel, thereby preventing ideal device operation.

The presented process is composed of two steps: spin coating and reflow. The spinning process removes any excess polyHEMA from the surface, and the thickness of polyHEMA layer can be precisely controlled by the spin speed. Although the rapid spinning speed may result in quicker evaporation of ethanol, leaving cavities on the surface and some trenches along the radial direction, these issues can be alleviated by reflowing the polyHEMA film at an elevated temperature after spin coating. As the glass transition temperature of polyHEMA is around 100° C., the polyHEMA film can reflow to fill the cavities and trenches at a temperature above 100° C. and below 200° C., the burning temperature of polyHEMA. The surface roughness root mean square (RMS) of the conventional coating process is more than 3 μm, while that of the spin-coated films is less than 0.2 μm. Compared to the conventional evaporation process, which generates high peaks and deep valleys, the thin and uniform polyHEMA films coated by the spin and reflow method can reliably bond with the PDMS layers for suspension cell culture.

PolyHEMA Lift-Off

In addition to uniform polyHEMA coating, lift-off technology was developed. First, the sacrificial layer was patterned on the surface, and then polyHEMA was spin-coated on the whole substrate. Then, the sacrificial layer was removed either by chemical etching or mechanical removal. The polyHEMA can be selectively patterned on the complement regions of the sacrificial layer regions.

PolyHEMA Stamping

The patterned stamp was fabricated by PDMS soft lithography. Then, a drop of polyHEMA in ethanol solution was put on top of the substrate. The stamp was then pressed from top, so the excess solution was squeezed out by the stamp. Then, the substrate and the stamp were placed on a hot plate and increase temperature to 100° C. The ethanol evaporates and leaks out through the PDMS, so the polyHEMA can be patterned on the substrate.

Mixing polyHEMA with Sensing Dyes as Micro-Sensors

Glucose, pH, oxygen and other sensing dyes can be mixed with PolyHEMA. Then, the dyes are patterned in the polyHEMA as a micro-sensor as small as 10 μm in a square pattern, allowing multiplexing of many sensors in an array. The sensor is then placed in the microfluidic chamber for the analysis of single cell metabolism.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, in vitro fertilization, development, or related fields are intended to be within the scope of the following claims.

1. A system, comprising:
a) a cell capture device comprising a plurality of wells, wherein each well is configured to capture a single cell, and wherein each well comprises a transport channel and a cell culture chamber;
b) a fluid transport device configured to transport fluid through the transport channel of said cell capture device without dead volume.

2. The system of claim 1, wherein said cell capture device comprises at least 100 wells.

3. The system of claim 1, wherein said cell capture device comprises at least 5000 wells.

4. The system of claim 1, wherein said cell capture device comprises at least 10,000 wells.

5. The system of claim 1, wherein said system further comprises a microscope in optical communication with said cell capture device.

6. The system of claim 1, wherein said fluid transport device is a vacuum device.

7. The system of claim 6, wherein said vacuum device and said cell culture chamber is approximately 50 μm.

8. The system of claim 1, wherein said cell capture device is constructed of PDMS.

9. The system of claim 1, wherein said cell culture well is approximately 100-500 μm in length and width.

10. The system of claim 1, wherein said main channel is approximately 50 μm in width.

11. The system of claim 1, wherein said cell capture device is approximately 40 to 100 μm in height.

12. The system of claim 1, wherein said system is configured to capture 10 or less cells present in a volume of 10 μl or less.

13. The system of claim 1, wherein said cell culture chamber further comprises one or more sensors for sensing pH, oxygen, glucose, proteins, or metabolic byproducts.

14. A method of capturing single cells, comprising:
a) contacting a solution comprising one or more cells with a system comprising a cell capture device comprising a plurality of wells, wherein each well is configured to capture a single cell, and wherein each well comprises a transport channel and a cell culture chamber; and a fluid transport device configured to transport fluid through the transport channel of said cell capture device without dead volume; and
b) transporting said solution through said system such that said cells are captured in said wells.

15. The method of claim 14, further comprising the step of contacting said cells with a test compound.

16. The method of claim 15, wherein said test compound is a drug.

17. A cell culture device, comprising a plurality of chambers, wherein at least one of each of said chambers comprises an adherent culture chamber configured for cells to adhere to, a suspension culture chamber configured to prevent adherence of cells, and a plurality of interaction channels.

18. The device of claim 17, wherein said suspension culture chamber is coated in polyHEMA.

19. The device of claim 18, wherein said polyHEMA is stamped and then reflowed at a temperature of between 100 and 200° C.

20. The device of claim 18, wherein said adherent culture chamber comprises a transwell and said suspension culture chamber comprises a plurality of microwells, wherein said microwells are coated in polyHEMA or Pluronic F108 surfactant.

21. The device of claim 18, wherein said suspension culture chamber and/or said adherent culture chamber comprise a plurality of bar code patterns for computer-aided tracking and/or monitoring.
